(19) United States
(12) Patent Application Publication
(10) Pub. No.: US 2008/0147092 A1
(43) Pub. Date: Jun. 19, 2008

(54) Hybrid energy instrument combined with clip application capability

(76) Inventors: Michael Rogge, Loveland, OH (US); Kevin L. Houser, Springboro, OH (US)

Correspondence Address: THOMPSON HINE L.L.P.;Intellectual Property Group, P.O. BOX 8801, DAYTON, OH, 45401-8801, US

(21) Appl. No.: 11/585,351

(22) Filed: Oct. 23, 2006

(51) Int. Cl. A61B 17/10

(52) U.S. Cl. 606/142

(57) ABSTRACT

A medical device including an elongated housing having a distal end, the housing defining a lumen channel therein, an end effector disposed at the distal end, the end effector including an energy-based surgical instrument having an elongated treatment portion and a clamp arm assembly, wherein the clamp arm assembly is moveable relative to the treatment portion, at least one clip disposed within the lumen channel and a firing bar movable through the lumen channel to advance the clip from a first position within the lumen channel to a second position external of the lumen channel.

### FIELD OF THE INVENTION

The present application relates generally to surgical instruments and methods and, more particularly, to methods and energy-based surgical instruments combined with clip application capability for cutting, coagulating and ligating tissue of a patient.

### BACKGROUND OF THE INVENTION

Surgeons typically use ultrasonic surgical devices to cut, coagulate and/or clot tissue. Exemplary ultrasonic surgical devices are described in U.S. Pat. Nos. 5,322,055, 6,325,811, 6,432,118 and 6,893,434, the entire contents of which are incorporated herein by reference. An example of an ultrasonic surgical device is the Harmonic Scalpel® Laparosonic® Coagulating Shears, available from Ethicon Endo-Surgery, Inc. of Cincinnati, Ohio.

In some procedures, an alternative instrument or an instrument exchange may be necessary to ligate or close tissue and/or vessels, thereby reducing the efficiency of the particular surgical procedures. Therefore, in order to prevent excessive fluid loss or bleeding during a surgical procedure, a surgeon will typically ligate, clamp or close the target tissue (e.g., a fluid duct or a blood vessel) before cutting.

There are many types of mechanisms or devices for clamping tissue, such as ligating clips and the like. Ligating clips have been formed of metal and may include a pair of legs that are connected at one end. The vessel to be ligated may be placed between the legs and the legs may be forced together about the vessel to close the vessel. Clips have also been developed from plastic materials. However, since plastics do not have the same strength and malleability as metals, plastic clips typically include some type of locking mechanism to secure the clips in a closed position.

Ligating clips should ensure closure of the vessel. That is, they should completely shut off blood flow or other fluid flow and not allow leakage. Also, the clips should remain closed, should not open or break and should not slip or slide out of position or off the vessel. An exemplary device is described in U.S. Pat. No. 5,921,997, the entire contents of which are incorporated herein by reference.

Clips typically are applied with a clip applier which crushes the clip to a preset dimension. Although a range of clip sizes exist to provide for ligation of a variety of tissue structure sizes, frequently the present dimension is too large for a smaller structure or too small for a larger structure, thereby mandating an instrument exchange to obtain the desired clip size to ensure closure of the vessel.

Energy based instruments for vessel sealing are also well known in the art and can utilize thermal, electrical, laser or ultrasound as their energy source. Frequently these instruments have a limited range of vessels that they are approved for sealing with large vessels of 4 mm or more being outside their approved range. When vessels of this size are encountered, the surgeon must remove the energy instrument from the surgical field and bring in either a clip applier or suture the vessel closed prior to transecting.

Accordingly, there is a need for an integration of clip applying capability to an energy-based surgical instrument to allow for eliminating instrument exchanges, decreasing potential of tissue damage during exchanges, and increasing the overall speed of surgical procedures.

### SUMMARY OF THE INVENTION

In one aspect, the disclosed medical device includes an elongated housing having a distal end, the housing defining a lumen channel therein, an end effector disposed at the distal end, the end effector including an energy-based surgical instrument having an elongated treatment portion and a clamp arm assembly, wherein the clamp arm assembly is moveable relative to the treatment portion, at least one clip disposed within the lumen channel and a firing bar movable through the lumen channel to advance the clip from a first position within the lumen channel to a second position external of the lumen channel.

In another aspect, the disclosed medical device includes an elongated housing having a distal end, the housing defining a lumen channel therein, an end effector disposed at the distal end, the end effector including an ultrasonic blade having an elongated treatment portion and a clamp arm assembly, wherein the clamp arm assembly is moveable relative to the treatment portion to grasp tissue positioned therebetween, an actuation member connected to the clamp arm assembly, the actuation member being adapted to move the clamp arm assembly from an open positioned to a closed position, at least one clip disposed within the lumen channel and a firing bar movable through the lumen channel to advance the clip from a first position within the lumen channel to a second position between the clamp arm assembly and the ultrasonic blade.

In another aspect, a method for treating a target tissue includes the steps of providing a hybrid medical device including a distal end having a clamp arm assembly and a energy-based surgical instrument portion disposed thereon, the energy-based surgical instrument portion including a treatment portion, positioning the distal end of the device adjacent to the target tissue, manipulating the distal end of the device to position the target tissue generally between the clamp arm assembly and the treatment portion, advancing a surgical clip from the device into a space between the clamp arm assembly and the treatment portion, crushing the clip over the target tissue and energizing the treatment portion to treat the target tissue.

Other aspects of the hybrid medical device and associated systems and methods will become apparent from the following detailed description, the accompanying drawings and the appended claims.

### BRIEF DESCRIPTION OF THE FIGURES

FIG. 1 is a side perspective view of a prior art ultrasonic surgical system;

FIG. 2 is a side perspective view of one aspect of the disclosed energy-based instrument combined with clip application capability;

FIG. 3 is a side elevational view, partially in section, of the device of FIG. 2;

FIG. 4 is an end elevational view of the device of FIG. 3;

FIG. 5 is a side perspective view of a second aspect of the disclosed energy-based instrument with clip application capability;

FIG. 6 is an end elevational view of the device of FIG. 5;

FIG. 7 is a side elevational view of a super-elastic clip closing over a vessel in accordance with another aspect of the disclosed energy-based instrument with clip application capability;

FIG. 7A is an end elevational view of the grasping jaw including a “T” shaped recess in accordance with another aspect of the disclosed energy-based instrument with clip application capability;

FIG. 8 is a side elevational view of a metallic clip having a protective elastomeric layer in accordance with another aspect of the disclosed energy-based instrument with clip application capability;

FIG. 9 is a perspective view of a thermally conductive clip in accordance with another aspect of the disclosed energy-based instrument with clip application capability;

FIG. 10 is a front elevational view of a snapover clip, in accordance with another aspect of the disclosed energy-based instrument with clip application capability; and

FIG. 11 is a front elevational view of a polymeric clip adapted with a tip weldable to itself with ultrasonic or other forms of energy in accordance with another aspect of the disclosed energy-based instrument with clip application capability.

### DETAILED DESCRIPTION OF THE INVENTION

Before describing the discloses hybrid medical device in detail, it should be noted that the disclosure is not limited in its application or use to the details of construction and arrangement of parts illustrated in the accompanying drawings and description. The illustrative embodiments may be implemented or incorporated in other embodiments, variations and modifications, and may be practiced or carried out in various ways. Furthermore, unless otherwise indicated, the terms and expressions employed herein have been chosen for the purpose of describing the illustrative embodiments for the convenience of the reader and are not intended to be limiting. Additionally, any embodiments shown to include a single type of energy source such as electrical or ultrasonic should not be assumed to be limited to that energy modality unless specifically stated.

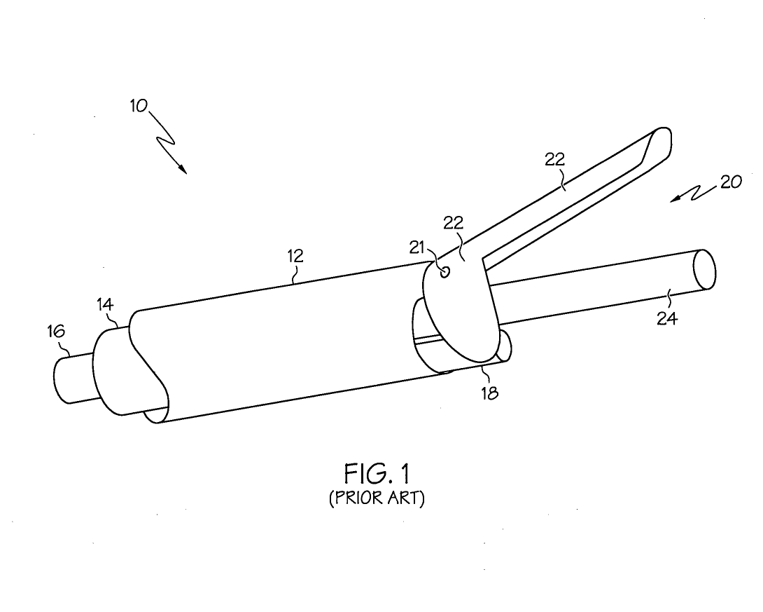

For purposes of comparison, a conventional ultrasonic surgical device, generally designated 10 in FIG. 1, typically includes an outer tube housing 12, an inner tube 14, a transmission component or ultrasonic waveguide 16 and an end-effector 20. The end effector 20 includes a clamp arm assembly 22 and an ultrasonic blade 24. In one configuration, the outer tube 12 may have a substantially circular cross-section and may be fabricated from surgical grade stainless steel. However, it will be recognized that the outer tube 12 may be constructed from any suitable material and may have any suitable cross-sectional shape. The clamp arm assembly 22 may be engagable by the distal end 18 of the inner tube 14 and be connected via pivot point 21 to outer tube 12 to allow it to pivot relative to the ultrasonic blade 24, thereby allowing a user to clamp tissue between the clamp arm 22 and the ultrasonic blade 24.

Ultrasonic vibrations are transmitted along the ultrasonic waveguide 16 in a longitudinal direction to vibrate the ultrasonic blade 24 at a predetermined vibrational frequency, however, torsional, transverse or some combination of the three vibrational modes may also be employed. The ultrasonic blade 24 may have a length substantially equal to an integral multiple of one-half system preset wavelengths (nλ/2). The ultrasonic blade 24 is preferably made from a solid core shaft constructed of material which propagates ultrasonic energy, such as a titanium alloy or an aluminum alloy. It will be recognized that the ultrasonic blade 24 may be fabricated from any other suitable material. It is also contemplated that the ultrasonic blade 24 may have a surface treatment to improve the delivery of energy and desired tissue effect. For example, the ultrasonic blade 24 may be micro-finished, coated, plated, etched, grit-blasted, roughened or scored to enhance coagulation and cutting of tissue and/or reduce adherence of tissue and blood to the end-effector. Additionally, the ultrasonic blade 24 may be sharpened or shaped to enhance its characteristics. Blade 24 may provide a multitude of edges and surfaces designed to provide a multitude of tissue effects: clamped coagulation, clamped cutting, grasping, back-cutting, dissection, spot coagulation, tip penetration and tip scoring.

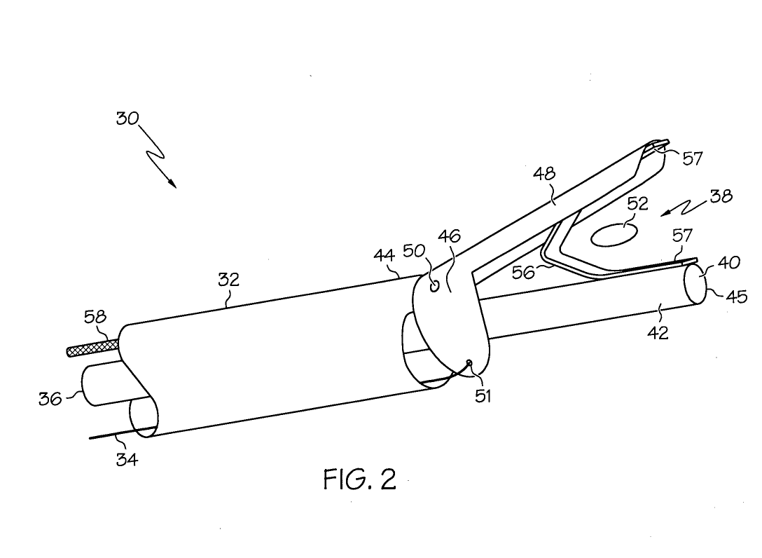

Referring to FIGS. 2-4, one aspect of the disclosed hybrid medical device, generally designated 30, may include an elongated housing 32, an actuation member 34, connected to a clamp arm assembly 46 at a connection point 51, a transmission component or ultrasonic waveguide 36 and an end-effector 38. The end-effector 38 may include an energy-based surgical instrument 40 and a clamp arm assembly 46 adapted to pivot relative to the instrument 40 at a pivot point 50. The instrument 40 may have a longitudinal axis and an elongated treatment portion 42 that may extend from the distal end 44 of the housing 32 to the tip 45 of the instrument 40. It is also conceivable that the elongated treatment portion may have a non-straightened shape such as a curve or multiple bends. The clamp arm assembly 46 may include a grasping jaw 48 adapted to move relative to the treatment portion 42 of the instrument 40 in response to movement of the actuation member 34 to clamp tissue 52 therebetween.

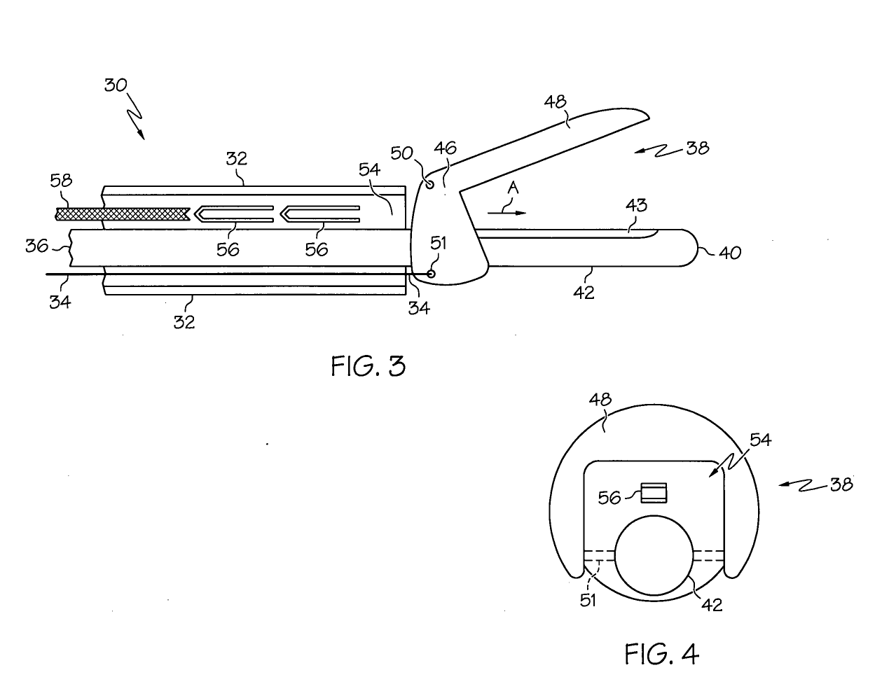

Those skilled in the art will appreciate that the actuation member 34 may be any technique or device capable of approximating the grasping jaw 48 towards the treatment portion 42 of the instrument and should not be limited to the actuation wire shown in FIGS. 2 and 3. For example, depending upon the structure and arrangement of the actuation member 34, a user may clamp tissue 52 between the jaw 48 and the treatment portion 42 by pushing (i.e., applying a force to the actuation member 34 in the distal direction), pulling (i.e., applying a force to the actuation member 34 in the proximal direction) or twisting (i.e., applying a rotational force to the actuation member 34) the actuation member 34.

The energy-based surgical instrument portion 40 of the energy-based surgical instrument 30 may be an ultrasonic vibrating blade, a radio frequency (RF) monopolar blade, a radio frequency bipolar blade, a laser fiber or an electrically resistive wire. The RF blade may be less expensive and may be easier to taper to a tissue-penetrating edge than using an ultrasonic blade in its place. However, RF blades may not be well suited for work around delicate structures where spread of the treatment energy is of concern. Consequently, ultrasonic blades may be more widely utilized in certain surgical procedures. In one aspect, the treatment portion 42 may be formed from or may include titanium, aluminum, ceramic, sapphire, or any other material that transmits ultrasound vibrations in an efficient manner.

Referring to FIGS. 3 and 4, a lumen channel 54 may be defined between the housing 32 and the waveguide 36 and one or more surgical clips 56 and a firing bar 58 may be position within the lumen channel 54. The surgical clips 56 may be partially nested and/or aligned within the lumen channel 54 such that, upon application of a force in the distal direction by the firing bar 58, a surgical clip 56 is ejected from the lumen channel 54 in the direction shown by arrow A (FIG. 3) between the jaw 48 and the treatment portion 42 to clip tissue 52 positioned and/or clamped therebetween.

Referring again to FIGS. 2-4, a clip 56 may be advanced from the lumen channel 54 into the end effector 38, as described above, and tissue 52 may be placed between the legs 57 of the clip 56 (see FIG. 2). The legs 57 may be forced together or crushed between the grasping jaw 48 and the treatment portion 42 to clamp the tissue 52 between the legs 57 of the clip 56. Optionally, ultrasonic energy may be passed from the waveguide 36 to the treatment portion 42 to coagulate, cut or otherwise treat the tissue 52 positioned within the clip 56 and/or between the jaw 48 and the treatment portion 42. Those skilled in the art will appreciate that the disclosed device 30 allows a skilled physician to apply ultrasonic (or other modality) energy before and/or after applying a clip 56 to tissue 52. In one embodiment of the current invention, clamping the clip 56 firmly between the treatment portion 42 and the jaw 48 allows the treatment potion 42 to ultrasonically couple with the clip 56, passing ultrasonic energy to the tissue 52 through the clip 56.

In one aspect, the device 30 may be sized to pass through a working channel of an endoscope (not shown). Furthermore, the size of the device 30 may be dictated by the size of the clip 56 as it is passed through the lumen channel 54 of the device 30 and/or the size of the grasping jaw 48 used to crush the clip 56. For example purposes only, the device 30 may be made from stainless steel if the device 30 will be reusable or polycarbonate if the device 30 will be disposable.

In another aspect, the clip 56 may be formed from titanium or any suitable alloy and preloaded into the lumen 54 in a slightly displaced condition. In this way, the clip 56 will spring open to a larger aperture after leaving the lumen 54 and moving into the end effector 38. It is also conceivable that the clip 56 could be made from a superelastic alloy such as Nitinol®.

In yet another embodiment, the surface of jaw 48 and/or the surface of treatment portion 42 that contacts the leg 57 of clip 56 includes a recessed section 43 that helps to guide the clip 56 into the end effector 38 and prevents the clip 56 from falling out of the end effector 38 until it is compressed.

Thus, device 30 provides an integration of clip applying capability (e.g., for ligating a blood vessel) with an energy-based surgical device (e.g., for coagulating tissue) in a single surgical instrument, thereby eliminating the need for instrument exchanges during a surgical procedure.

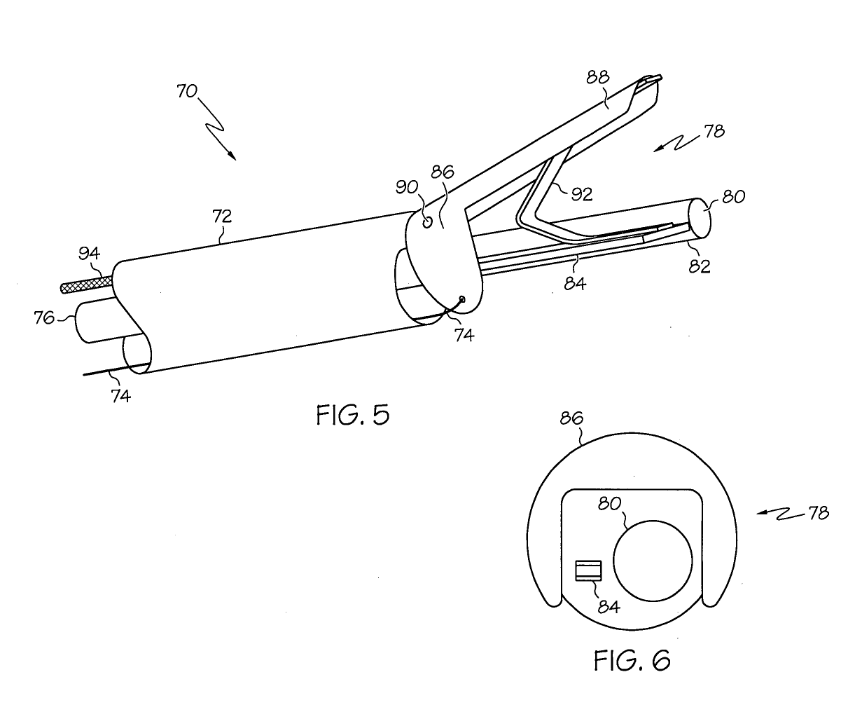

Referring to FIGS. 5 and 6, another aspect of the disclosed hybrid medical device, generally designated 70, may include a housing 72, an actuation member 74, a transmission component or ultrasonic waveguide 76 and an end-effector 78. The end-effector 78 may include an energy-based surgical instrument 80 having a treatment portion 82, an anvil 84 positioned generally adjacent to the treatment portion 82 and a clamp arm assembly 86 having a grasping jaw 88. The clamp arm assembly 86 may be adapted to pivot relative to the anvil 84 at a pivot point 90 such that the grasping jaw 88 may approximate the anvil 84 and the grasping jaw 88 to clamp tissue (not shown) positioned therebetween.

As discussed above with respect to device 30, a clip 92 may be positioned within the housing 72 and ejected from the housing by a firing rod 94 such that the clip 92 is positioned between the grasping jaw 88 and the anvil 84, which is adjacent to the treatment portion 82 of the energy-based surgical instrument 80. Therefore, as discussed above, the device 70 allows a physician to clamp, clip and/or coagulate tissue with a singe device, without using the sensitive and delicate treatment portion 82 of the energy-based instrument 80 as an anvil. As stated above, jaw 88 or anvil 84 may also include a recess (not shown) to guide the clip as it is positioned therebetween.

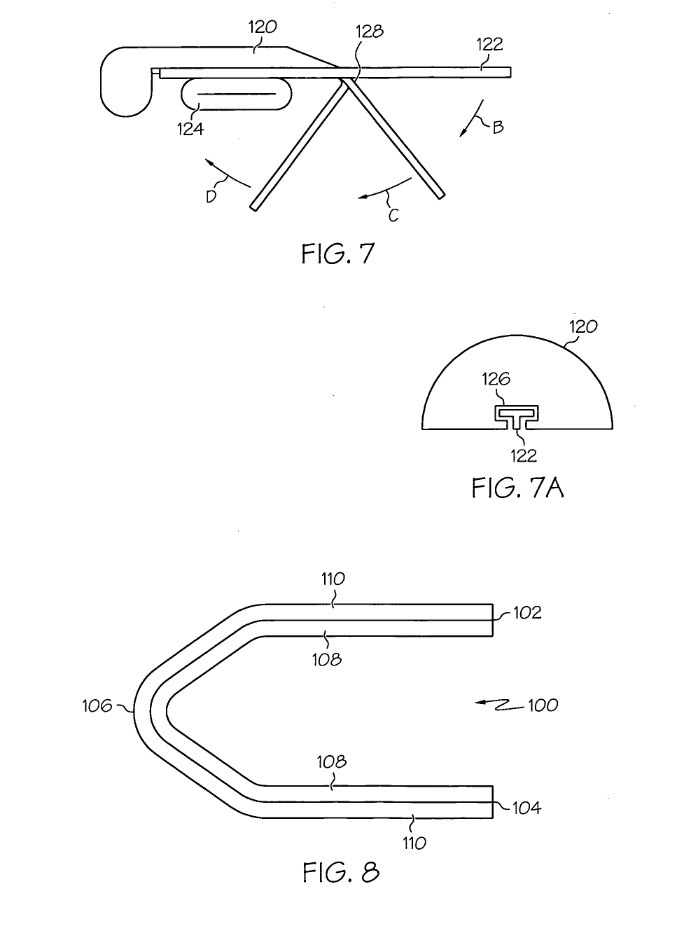

Referring to FIGS. 7-11, various clips may be used with the disclosed hybrid devices.

For example, referring to FIG. 7, with the grasping jaw 120 of the disclosed hybrid device in a closed position, a superelastic clip 122 may be passed through the lumen channel (not shown) in an open position. The body temperature of the patient and/or heat from the treatment portion 42 may be sufficiently high to bring the clip 122 to its transition temperature such that the clip returns to its original shape (e.g., a clip shape) (see arrows B, C and D) and clamps tissue 124 grasped by the grasping jaw 120. In one aspect, the clip 122 may be formed from Nitinol (i.e., nickel/titanium alloys) or any other shape memory material or superelastic material.

In another embodiment, as shown in FIG. 7A, the clip 122, is manufactured of a superelastic alloy and is pre-bent into a straight shape and loaded into the instrument. Clip 122 also has a “T” shaped cross section. Jaw 120 includes a “T” shaped recess 126 and as clip 122 is advanced through jaw 120, the pre-bent position 128 of clip 122 comes free of jaw 120, allowing the clip 122 to return to its undeformed shape and compress tissue 124.

Referring to FIG. 8, a clip, generally designated 100, may include a first leg portion 102 connected to a second leg portion 104 at a connection portion 106. The clip 100 may include a first, inner layer 108 and a second, outer layer 110, wherein the inner layer 108 may be formed from a formable material, such as stainless steel, and the outer layer 110 may be formed from a soft or elastomeric material, such as rubber. The outer layer 110 may prevent scratching or other damage to the treatment portion 42 of the energy-based surgical instrument 40 when the clip 100 is ejected from the housing lumen channel 54 as described above.

In another embodiment, inner layer 108 and outer layer 110 may or may not both be continuous, i.e., inner layer 108 or outer layer 110 could be formed such that one does not extend fully distally to the tips of the legs 102 and 104 of clip 100 or fully proximally to the connection portion 106 of clip 100. Inner layer 108 or outer layer 110 may also be made up of sections equally spaced along legs 102 and 104. Additionally, inner layer 108 or outer layer 110 may or may not be disposed on both legs 102 and 104 of clip 100. Outer layer 110 may also fully encapsulate inner layer 108.

In another embodiment, inner layer 108 may be made from a soft or elastomeric material such as rubber and outer layer 110 may be made from a formable material such as stainless steel. Thus when clip 100 is compressed around tissue, the soft inner layer 108 maintains compression on the tissue 124 over a wide range of compression and closure on clip 100.

In another embodiment, outer layer 110 and inner layer 108 of clip 100 are selected from materials based on but not limited to one or more material properties of electrical conductivity, thermal conductivity, elastic modulus, tensile strength, yield strength, porosity, surface finish or melt point. One or both of layers 108 and 110 may be porous. These porous structures may contain but are not limited to one or more of thrombic agents to promote clotting, antibiotics, anti-inflammatory agents or immune suppressive agents. Inner layer 108 and/or outer layer 110 may also be made of materials with antibacterial properties or could be coated with such materials.

Referring to FIG. 9, there is illustrated a clip, generally designated 130, which may be a wide material with good thermal transfer properties. It may be possible to widen the thermally affected zone 134 of the tissue 132 over the thermally affected zone 136 created by the ultrasonic instrument alone by closing the wide clip 130 over tissue 132 and applying ultrasonic energy to heat the clip 130. There may be applications where a wider heat affected zone 134 is advantageous for sealing many small vessels at once. For example, addressing many small vessels one at a time within a large transected area may be time-consuming and/or impractical for the surgeon or instrument user.

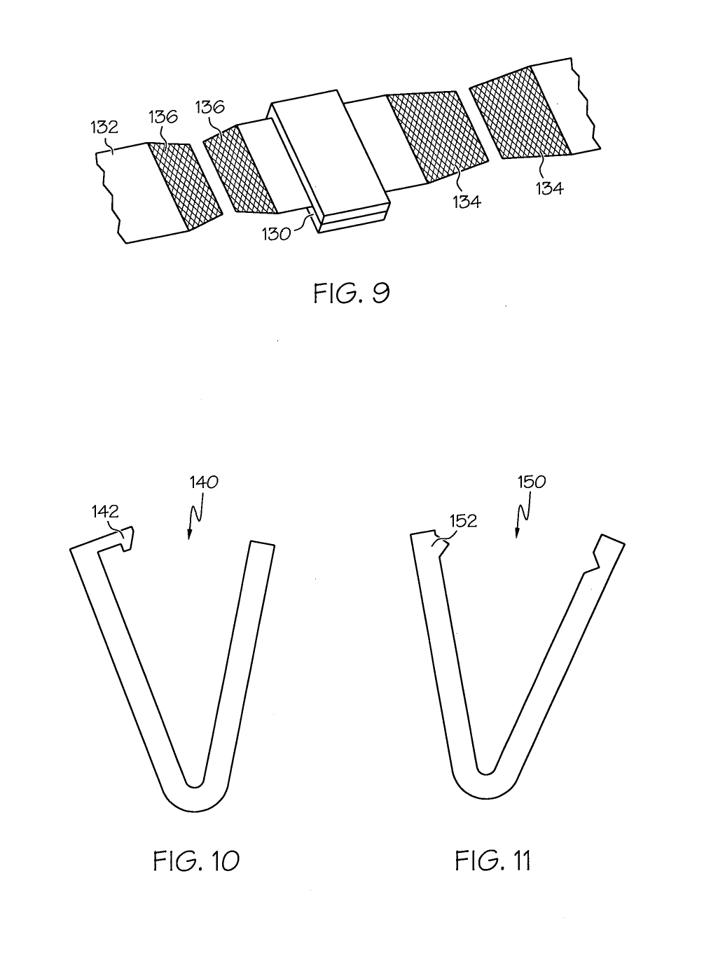

Referring to FIG. 10, there is illustrated a clip, generally designated 140, that includes a distal snap 142 that locks the clip 140 in a closed configuration about a vessel.

Referring to FIG. 11, there is illustrated a clip, generally designated 150, made from a polymeric material adapted to be melted at the ends 152 by, for example, ultrasonic or RF welding techniques, thereby securing the clip 150 in a closed configuration.

The clips described herein may be made from various well-known materials or alloys of materials, such as, for example, titanium, tantalum, stainless steel, memory metals having super-elastic characteristics or the various plastic materials that have some resiliency (i.e., a predetermined minimum yield strength) such as polyolefins, polycarbonates, glycolide-lactide polymers and similar plastic materials.

In one aspect a method for treating patient tissue may include the steps of: a) providing a medical device adapted for treating patient tissue including a distal end having a clamp arm assembly and a energy-based surgical instrument further including a treatment portion; b) inserting said medical device distal end into the patient; c) manipulating said medical device distal end to capture and ligate said tissue; d) advancing a surgical clip into the space between said clamp arm assembly and said treatment portion of said energy-based surgical instrument; e) crushing said clip to capture and occlude said tissue; f) manipulating said medical device distal end to coagulate said tissue; and g) withdrawing said medical device distal end from the patient.

In another aspect a method for treating patient tissue may include the steps of: a) providing a medical device adapted for treating patient tissue including a distal end having a clamp arm assembly and a energy-based surgical instrument further including a treatment portion; b) inserting said medical device distal end into the patient; c) advancing a surgical clip into the space between said clamp arm assembly and said treatment portion of said energy-based surgical instrument; d) manipulating said medical device distal end to capture and clip said tissue; e) crushing said clip to capture and occlude said tissue; f) manipulating said medical device distal end to ligate said tissue; and g) withdrawing said medical device distal end from the patient.

The foregoing description has been presented for purposes of illustration. It is not intended to be exhaustive or limiting and obviously many modifications and variations are possible in light of the above teaching. For example, as would be apparent to those skilled in the art, the disclosures herein of the hybrid medical device have equal application in robotic assisted surgery taking into account the obvious modifications of such systems and components to be compatible with such a robotic system.

What is claimed is:

1. A medical device comprising:
an elongated housing having a distal end, said housing defining a lumen channel therein;
an end effector disposed at said distal end, said end effector including an energy-based surgical instrument having an elongated treatment portion and a clamp arm assembly, wherein said clamp arm assembly is moveable relative to said treatment portion;
at least one clip disposed within said lumen channel; and
a firing bar movable through said lumen channel to advance said clip from a first position within said lumen channel to a second position external of said lumen channel.

2. The medical device of claim 1 wherein said energy-based surgical instrument is an ultrasonic blade.

3. The medical device of claim 2 wherein said ultrasonic blade vibrates in a longitudinal mode.

4. The medical device of claim 2 wherein said ultrasonic blade vibrates in a torsional mode.

5. The medical device of claim 2 wherein said ultrasonic blade vibrates in a transverse mode.

6. The medical device of claim 1 wherein said energy-based surgical instrument is an RF blade.

7. The medical device of claim 1 wherein said energy-based surgical instrument is a laser fiber.

8. The medical device of claim 1 wherein said energy-based surgical instrument is an electrically resistive wire heated by electrical current passed through it.

9. The medical device of claim 1 wherein said clamp arm assembly includes a grasping jaw adapted to grasp tissue positioned between said clamp arm assembly and said treatment portion.

10. The medical device of claim 1 wherein said clamp arm assembly is pivotally connected to said treatment portion.

11. The medical device of claim 1 further comprising an actuation member connected to said clamp arm assembly.

12. The medical device of claim 8 wherein actuation of said actuation member moves said clamp arm assembly towards said treatment portion.

13. The medical device of claim 1 further comprising an ultrasonic waveguide extending though said housing and connected to said energy-based surgical instrument.

14. The medical device of claim 1 wherein said clip is formed from a shape memory material and is disposed within said lumen channel in an open position.

15. The medical device of claim 14 wherein said shape memory material is a titanium/nickel alloy.

16. The medical device of claim 1 wherein said clip includes a first leg member, a second leg member and a connection portion, said first leg member being connected to said second leg member at said connection portion.

17. The medical device of claim 1 wherein said clip is coated with a soft, elastomeric material.

18. The medical device of claim 16 wherein said first leg includes a distal snap adapted to engage a distal portion of said second leg.

19. The medical device of claim 1 wherein, when said clip is in said second position, said clip is positioned between said clamp arm assembly and said treatment portion.

20. The medical device of claim 1 wherein said end effector further includes an anvil, said anvil being positioned generally adjacent to and generally parallel with said treatment portion.

21. The medical device of claim 15 wherein, when said clip is in said second position, said clip is positioned between said clamp arm assembly and said anvil.

22. A medical device comprising:
an elongated housing having a distal end, said housing defining a lumen channel therein;
an end effector disposed at said distal end, said end effector including an ultrasonic blade having an elongated treatment portion and a clamp arm assembly, wherein said clamp arm assembly is moveable relative to said treatment portion to grasp tissue positioned therebetween;
an actuation member connected to said clamp arm assembly, said actuation member being adapted to move said clamp arm assembly from an open positioned to a closed position;
at least one clip disposed within said lumen channel; and
a firing bar movable through said lumen channel to advance said clip from a first position within said lumen channel to a second position between said clamp arm assembly and said ultrasonic blade.

23. The medical device of claim 22 wherein said clamp arm assembly includes a grasping jaw adapted to grasp tissue positioned between said clamp arm assembly and said ultrasonic blade.

24. The medical device of claim 22 wherein said end effector further includes an anvil, said anvil being positioned generally adjacent to, and generally parallel with, said treatment portion.

25. A method for treating a target tissue comprising the steps of:
providing a hybrid medical device including a distal end having a clamp arm assembly and a energy-based surgical instrument portion disposed thereon, said energy-based surgical instrument portion including a treatment portion;
positioning said distal end of said device adjacent to said target tissue;
manipulating said distal end of said device to position said target tissue generally between said clamp arm assembly and said treatment portion;
advancing a surgical clip from said device into a space between said clamp arm assembly and said treatment portion;
crushing said clip over said target tissue; and
energizing said treatment portion to treat said target tissue.

26. A method for treating a target tissue comprising the steps of:
providing a hybrid medical device including a distal end having a clamp arm assembly and a energy-based surgical instrument portion disposed thereon, said energy-based surgical instrument portion including a treatment portion;
positioning said distal end of said device adjacent to said target tissue;
manipulating said distal end of said device to position said target tissue generally between said clamp arm assembly and said treatment portion to capture and ligate said target tissue;
advancing a surgical clip from said device into a space between said clamp arm assembly and said treatment portion;
crushing said clip over said target tissue; and
energizing said treatment portion to treat said target tissue.
